SEQUENCE LISTING (24 sequences)

SEQ ID NO: 1 - protein, 215 aa, Artificial Sequence
  AX213 Antibody Full Light Chain
EIVLTQSPATLSLSPGERATITCRASQYVGSYLNWYQQKPGQAPRLLIYDASNRATGIPARFSGSGSGTDFTLTISSLEPEDFAVYYCQVWDSSPPVVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - DNA, 645 bp, Artificial Sequence
  AX213 Antibody Full Light Chain
gaaatcgtgctgacccagtctccagccaccctgtctctgtctcccggggaacgtgccaccatcacctgccgtgcctctcagtatgtcggcagctacctgaactggtatcagcagaagccaggtcaggcgccacgtctgctgatctacgacgcctctaaccgtgccaccggtatcccagcccgtttctctggttctggttctggcaccgacttcaccctgaccatctcttctctggaaccagaagacttcgccgtgtactactgccaggtatgggacagctctcctcctgtggtgttcggtggtggtaccaaagtggaaatcaagcgtacggtggctgcaccatctgtattcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 3 - protein, 108 aa, Artificial Sequence
  AX213 Variable Light Antibody Region
EIVLTQSPATLSLSPGERATITCRASQYVGSYLNWYQQKPGQAPRLLIYDASNRATGIPARFSGSGSGTDFTLTISSLEPEDFAVYYCQVWDSSPPVVFGGGTKVEIK

SEQ ID NO: 4 - DNA, 324 bp, Artificial Sequence
  AX213 Variable Light Antibody Region
gaaatcgtgctgacccagtctccagccaccctgtctctgtctcccggggaacgtgccaccatcacctgccgtgcctctcagtatgtcggcagctacctgaactggtatcagcagaagccaggtcaggcgccacgtctgctgatctacgacgcctctaaccgtgccaccggtatcccagcccgtttctctggttctggttctggcaccgacttcaccctgaccatctcttctctggaaccagaagacttcgccgtgtactactgccaggtatgggacagctctcctcctgtggtgttcggtggtggtaccaaagtggagatcaaa

SEQ ID NO: 5 - protein, 226 aa, Artificial Sequence
  AX213 Antibody Fd Chain
QVQLLESGGGLVQPGGSLRLSCKASGYTFSRYGINWVRQAPGKGLEWIGRIDPGNGGTRYNEKFKGKATISRDNSKNTLYLQMNSLRAEDTAVYYCARANDGYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 6 - DNA, 678 bp, Artificial Sequence
  AX213 Antibody Fd Chain
caggtgcaattgctggaatctggtggtggtctggtgcagccaggtggttctctgcgtctgtcttgcaaggctagcggttacaccttctctcgctacggtatcaactgggtgcgtcaggcaccaggtaagggtctggaatggatcggtcggatcgacccaggtaacggtggtactaggtacaacgaaaagttcaagggtaaggccaccatctctagagacaactctaagaacaccctgtacttgcagatgaactctctgcgtgccgaggacactgcagtgtactactgcgcccgtgcaaatgacggttactccttcgactactggggtcagggtacgctggtgactgtctcgagcgcaagcaccaaaggcccatcggtattccccctggcaccctcctccaagagcacctctgggggcacagcggccctgggctgcctggtcaaggactacttccccgagccggtgacggtgtcgtggaactcaggcgctctgaccagcggcgtgcacaccttcccggctgtcctacagtcctcaggactctactccctcagcagcgtggtgactgtgccctccagcagcttgggcacccagacctacatctgcaacgtgaatcacaagcccagcaacactaaggtggacaagaaagttgagcccaaatcttgtgacaaaactcacaca

SEQ ID NO: 7 - protein, 118 aa, Artificial Sequence
  AX213 Antibody Variable Heavy Region
EVQLLESGGGLVQPGGSLRLSCKASGYTFSRYGINWVRQAPGKGLEWIGRIDPGNGGTRYNEKFKGKATISRDNSKNTLYLQMNSLRAEDTAVYYCARANDGYSFDYWGQGTLVTVSS

SEQ ID NO: 8 - DNA, 354 bp, Artificial Sequence
  AX213 Antibody Variable Heavy Region
caggtgcaattgctggaatctggtggtggtctggtgcagccaggtggttctctgcgtctgtcttgcaaggctagcggttacaccttctctcgctacggtatcaactgggtgcgtcaggcaccaggtaagggtctggaatggatcggtcggatcgacccaggtaacggtggtactaggtacaacgaaaagttcaagggtaaggccaccatctctagagacaactctaagaacaccctgtacttgcagatgaactctctgcgtgccgaggacactgcagtgtactactgcgcccgtgcaaatgacggttactccttcgactactggggtcagggtacgctggtgactgtctcgagc

SEQ ID NO: 9 - protein, 444 aa, Artificial Sequence
  AX213 Antibody Heavy Chain
EVQLLESGGGLVQPGGSLRLSCKASGYTFSRYGINWVRQAPGKGLEWIGRIDPGNGGTRYNEKFKGKATISRDNSKNTLYLQMNSLRAEDTAVYYCARANDGYSFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - DNA, 1332 bp, Artificial Sequence
  AX213 Antibody Heavy Chain
gaggtccaacttttggagtctggaggaggactggtccaacctggaggctccctgagactgtcctgtaaggcatctggctacaccttcagcagatatggcatcaactgggtgagacaggctcctggcaagggattggagtggattggcaggattgaccctggcaatggaggcaccagatacaatgagaagttcaagggcaaggctaccatcagcagggacaacagcaagaacaccctctacctccaaatgaactccctgagggctgaggacacagcagtctactactgtgccagggctaatgatggctactcctttgactactggggacaaggcaccctggtgacagtgtcctctgctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 11 - protein, 215 aa, Artificial Sequence; identical to SEQ ID NO: 1
  AX213 Antibody Light Chain

SEQ ID NO: 12 - DNA, 645 bp, Artificial Sequence
  AX213 Antibody Light Chain
gagattgtgctgacccagagccctgccaccctgtccctgagccctggagagagggctaccatcacttgtagggcaagccaatatgtgggctcctacctgaactggtatcaacagaagcctggacaagccccaagactgctgatttatgatgccagcaacagggctacaggcatccctgccaggttctctggctctggctctggcacagacttcaccctgaccatctcctccttggaacctgaggactttgctgtctactactgtcaggtgtgggactccagccctcctgtggtgtttggaggaggcaccaaggtggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 13 - protein, 216 aa, Artificial Sequence
  AX1 Antibody Light Chain
DIQMTQSPSSLSASVGDRVTITCRASQDISRYLAWYQQKPGKAPKLLIYAASSLQSGVPSRFSGSGSGTDFTLTISSLQPEDFATYYCAAYDYSLGGYVFGDGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 648 bp, Artificial Sequence
  AX1 Antibody Light Chain
gacatccagatgacccagtctccatcttctctgtctgcctctgtgggcgaccgggtgaccatcacctgccgtgcctctcaggatatctctaggtatctggcctggtatcagcagaagccaggtaaggcgccaaagctgctgatctacgccgcctcttctttgcagtctggtgtgccatctcgtttctctggttctggttctggcaccgacttcaccctgaccatctcttctttgcagccagaagacttcgccacctactactgcgcggcttacgactattctttgggcggttacgtgttcggtgatggtaccaaagtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 15 - protein, 109 aa, Artificial Sequence
  AX1 Antibody Variable Light Region
DIQMTQSPSSLSASVGDRVTITCRASQDISRYLAWYQQKPGKAPKLLIYAASSLQSGVPSRFSGSGSGTDFTLTISSLQPEDFATYYCAAYDYSLGGYVFGDGTKVEIK

SEQ ID NO: 16 - DNA, 327 bp, Artificial Sequence
  AX1 Antibody Variable Light Region
gacatccagatgacccagtctccatcttctctgtctgcctctgtgggcgaccgggtgaccatcacctgccgtgcctctcaggatatctctaggtatctggcctggtatcagcagaagccaggtaaggcgccaaagctgctgatctacgccgcctcttctttgcagtctggtgtgccatctcgtttctctggttctggttctggcaccgacttcaccctgaccatctcttctttgcagccagaagacttcgccacctactactgcgcggcttacgactattctttgggcggttacgtgttcggtgatggtaccaaagtggagatcaaa

SEQ ID NO: 17 - protein, 227 aa, Artificial Sequence
  AX1 Antibody Fd Chain
EVQLLESGGGLVQPGGSLRLSCKASGFTFTSYYMHWVRQAPGKGLEWIGRINPDSGSTKYNEKFKGRATISRDNSKNTLYLQMNSLRAEDTAVYYCARGGRLSWDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 18 - DNA, 681 bp, Artificial Sequence
  AX1 Antibody Fd Chain
gaagtgcagctgctggaatctggtggtggtctggtgcagccaggtggttctctgcgtctgtcttgcaaggcctctggtttcaccttcacttcttactacatgcactgggtgcgtcaggcaccaggtaagggtctggaatggatcggtcggatcaacccagattctggtagtactaagtacaacgagaagttcaagggtcgtgccaccatctctagagacaactctaagaacaccctgtacttgcagatgaactctctgcgtgccgaggacactgcagtgtactactgcgcccgtggtggtcgtttatcctgggacttcgacgtctggggtcagggtacgctggtgactgtctcgagcgcaagcaccaaaggcccatcggtattccccctggcaccctcctccaagagcacctctgggggcacagcggccctgggctgcctggtcaaggactacttccccgagccggtgacggtgtcgtggaactcaggcgctctgaccagcggcgtgcacaccttcccggctgtcctacagtcctcaggactctactccctcagcagcgtggtgactgtgccctccagcagcttgggcacccagacctacatctgcaacgtgaatcacaagcccagcaacactaaggtggacaagaaagttgagcccaaatcttgtgacaaaactcacaca

SEQ ID NO: 19 - protein, 119 aa, Artificial Sequence
  AX1 Antibody Variable Heavy Region
EVQLLESGGGLVQPGGSLRLSCKASGFTFTSYYMHWVRQAPGKGLEWIGRINPDSGSTKYNEKFKGRATISRDNSKNTLYLQMNSLRAEDTAVYYCARGGRLSWDFDVWGQGTLVTVSS

SEQ ID NO: 20 - DNA, 357 bp, Artificial Sequence
  AX1 Antibody Variable Heavy Region
gaagtgcagctgctggaatctggtggtggtctggtgcagccaggtggttctctgcgtctgtcttgcaaggcctctggtttcaccttcacttcttactacatgcactgggtgcgtcaggcaccaggtaagggtctggaatggatcggtcggatcaacccagattctggtagtactaagtacaacgagaagttcaagggtcgtgccaccatctctagagacaactctaagaacaccctgtacttgcagatgaactctctgcgtgccgaggacactgcagtgtactactgcgcccgtggtggtcgtttatcctgggacttcgacgtctggggtcagggtacgctggtgactgtctcgagc

SEQ ID NO: 21 - protein, 445 aa, Artificial Sequence
  AX1 Antibody Heavy Chain
EVQLLESGGGLVQPGGSLRLSCKASGFTFTSYYMHWVRQAPGKGLEWIGRINPDSGSTKYNEKFKGRATISRDNSKNTLYLQMNSLRAEDTAVYYCARGGRLSWDFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - DNA, 1335 bp, Artificial Sequence
  AX1 Antibody Heavy Chain
gaggtccaacttttggagtctggaggaggactggtccaacctggaggctccctgagactgtcctgtaaggcatctggcttcaccttcacctcctactatatgcactgggtgagacaggctcctggcaagggattggagtggattggcaggataaaccctgactctggcagcaccaaatacaatgagaagttcaagggcagggctaccatcagcagggacaacagcaagaacaccctctacctccaaatgaactccctgagggctgaggacacagcagtctactactgtgccaggggaggcagactgtcctgggactttgatgtgtggggacaaggcaccctggtgacagtgtcctctgctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 23 - protein, 216 aa, Artificial Sequence; identical to SEQ ID NO: 13
  AX1 Antibody Light Chain

SEQ ID NO: 24 - DNA, 648 bp, Artificial Sequence
  AX1 Antibody Light Chain
gacatccagatgacccagagcccatcctccctgtctgcctctgtgggagacagggtgaccatcacttgtagggcaagccaggacatcagcagatacctggcttggtatcaacagaagcctggcaaggctccaaaactgctgatttatgctgcctcctccctccaatctggagtgccaagcaggttctctggctctggctctggcacagacttcaccctgaccatctcctccctccaacctgaggactttgccacctactactgtgctgcctatgactactccctgggaggctatgtgtttggagatggcaccaaggtggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2012/0208209 A1
(43) Pub. Date: Aug. 16, 2012

(54) PCSK9 IMMUNOASSAY

(76) Inventors: Marina Ichetovkin, Highland Park, NJ (US); Zhu Chu, Edison, NJ (US); Cheryl Le Grand, Langhorne, PA (US)

(21) Appl. No.: 13/503,732

(22) PCT Filed: Oct. 29, 2010
(86) PCT No.: PCT/US2010/054595

§ 371 Date: Apr. 24, 2012

Related U.S. Application Data
(60) Provisional application No. 61/256,752, filed on Oct. 30, 2009.
(60) Provisional application No. 61/368,081, filed on Jul. 27, 2010.

(51) Int. Cl. G01N 33/573; C12M 1/34

(52) U.S. Cl. 435/7.4; 435/287.2

(57) ABSTRACT

Methods of using PCSK9 antagonists. More specifically, methods for measuring circulating PCSK9 levels in a biological sample by means of an immunoassay.

### BACKGROUND OF THE INVENTION

Proprotein convertase subtilisin-kexin type 9 (PCSK9), also known as neural apoptosis-regulated convertase 1 (NARC-1), is a proteinase K-like subtilase identified as the 9^{th} member of the secretory subtilase family (Seidah, N. G., et al., 2003 PROC NATL ACAD SCI USA 100:928-933). PCSK9 is expressed in cells capable of proliferation and differentiation such as hepatocytes, kidney mesenchymal cells, intestinal ileum, colon epithelia and embryonic brain telencephalic neurons (Seidah et al., 2003).

The gene for human PCSK9 has been sequenced and found to be about 22-kb long with 12 exons that encode a 692 amino acid protein (NP_{—}777596.2). PCSK9 is disclosed and/or claimed in several patent publications, including: PCT Publication Nos. WO 01/31007, WO 01/57081, WO 02/14358, WO 01/98468, WO 02/102993, WO 02/102994, WO 02/46383, WO 02/90526, WO 01/77137, and WO 01/34768; US Publication Nos. US 2004/0009553 and US 2003/0119038, and European Publication Nos. EP 1 440 981, EP 1 067 182, and EP 1 471 152.

PCSK9 has been implicated in cholesterol homeostasis, as it appears to have a specific role in cholesterol biosynthesis or uptake. In a study of cholesterol-fed rats, Maxwell et al. found that PCSK9 was downregulated in a similar manner to other genes involved in cholesterol biosynthesis, (Maxwell et al. 2003 J. LIPID RES. 44:2109-2119). The expression of PCSK9 was regulated by sterol regulatory element-binding proteins (SREBP), which is seen in other genes involved in cholesterol metabolism (Maxwell, et al., 2003).

Additionally, PCSK9 expression is upregulated by statins in a manner attributed to the cholesterol-lowering effects of the drugs (Dubuc et al., 2004 ARTERIOSCLER. THROMB. VASO. BIOL. 24:1454-1459). Adenoviral expression of PCSK9 has been shown to lead to a notable time-dependent increase in circulating low density lipoprotein (LDL) (Benjannet et al., 2004 J. BIOL. CHEM. 279:48865-48875) and mice with PCSK9 gene deletions have increased levels of hepatic LDL receptors (LDLR) and clear LDL from the plasma more rapidly (Rashid et al., 2005 PROC. NATL. ACAD. Sci. USA 102:5374-5379). Medium from HepG2 cells transiently transfected with PCSK9 reduce the amount of cell surface LDLRs and internalization of LDL when transferred to untransfected HepG2 cells (Cameron et al., 2006 HUMAN MOL. GENET. 15:1551-1558). It has been further demonstrated that purified PCSK9 added to the medium of HepG2 cells had the effect of reducing the number of cell-surface LDLRs in a dose- and time-dependent manner (Lagace et al., 2006 J. CLIN. INVEST. 116:2995-3005).

A number of mutations in the gene PCSK9 have also been conclusively associated with autosomal dominant hypercholesterolemia (ADH), an inherited metabolism disorder characterized by marked elevations of low density lipoprotein (“LDL”) particles in the plasma which can lead to premature cardiovascular failure (e.g., Abifadel et al., 2003 NATURE GENETICS 34:154-156; Timms et al., 2004 HUM. GENET. 114:349-353; Leren, 2004 CLIN. GENET. 65:419-422).

It therefore appears that PCSK9 plays a role in the regulation of LDL production. Expression or upregulation of PCSK9 is associated with increased plasma levels of LDL cholesterol, and inhibition or the lack of expression of PCSK9 is associated with low LDL cholesterol plasma levels. Significantly, lower levels of LDL cholesterol associated with sequence variations in PCSK9 confer protection against coronary heart disease (Cohen, et al., 2006N. ENGL. J. MED. 354:1264-1272).

Clinical trial data have demonstrated that reductions in LDL cholesterol levels are related to the rate of coronary events (Law et al., 2003 BMJ 326:1423-1427). Moderate lifelong reduction in plasma LDL cholesterol levels has been shown to be substantially correlated with a substantial reduction in the incidence of coronary events (Cohen et al., 2006, supra), even in populations with a high prevalence of non-lipid-related cardiovascular risk factors. Accordingly, there is great benefit to be reaped from the managed control of LDL cholesterol levels.

Accordingly, it would be desirable to further investigate PCSK9 as a target for the treatment of cardiovascular disease. Antibodies useful as PCSK9 antagonists have been identified and have utility as therapeutic agents. In support of such investigations, it would be useful to have a method for measuring levels of circulating PCSK9 in a biological sample which has been exposed to a PCSK9 antagonist, such as an antibody.

It would be further desirable to be able to identify novel PCSK9 antagonists in order to assist in the quest for compounds and/or agents effective in the treatment of cardiovascular disease. Hence, a method for measuring levels of circulating PCSK9 in a biological sample for such purposes as, e.g., assessing the effectiveness of a putative PCSK9 antagonist is desirable.

Additionally, it would be of use to provide kits to assay levels of circulating PCSK9 in biological samples.

### SUMMARY OF THE INVENTION

The present invention relates to a method of measuring circulating PCSK9 levels in a biological sample. Said method comprises the steps of performing an immunoassay on a biological sample obtained from a subject and comparing the level of PCSK9 in said sample against a standard having a known concentration of PCSK9.

The present invention further relates to a method for identifying novel PCSK9 antagonists, comprising the steps of performing an immunoassay on a biological sample which has been contacted with a putative PCSK9 antagonist and comparing the level of PCSK9 in said sample against a standard having a known concentration of PCSK9.

A further aspect of the present invention relates to a kit for measuring circulating PCSK9 levels in a biological sample, wherein said kit comprises:

a). a biological sample collection device;

b). a composition comprising an immunoassay which comprises a coating or capture antibody and a detection antibody;

and c). a means for detecting a reaction between PCSK9 antigen in the sample and antibodies in the immunoassay.

### BRIEF DESCRIPTION OF THE DRAWINGS

FIGS. 1A-B illustrates the Lanthanide Chelate Delay time and Stokes' shift

FIG. 2 illustrates the recombinant human PCSK9 standard curve diluted in assay buffer. The range of the curve is 10.26 nM to 0.005 nM.

FIG. 3 illustrates the biological variability of six normal healthy volunteers shown on three different days over three weeks. Concentration shown in nM.

### DETAILED DESCRIPTION OF THE INVENTION

The present invention relates to a method of measuring circulating PCSK9 levels in a biological sample, comprising the steps of performing an immunoassay on a biological sample obtained from a subject and comparing the level of PCSK9 in said sample against a standard having a known concentration of PCSK9. The present assay is of particular utility for measuring human PCSK9.

An immunoassay is an analysis or methodology that utilizes an antibody to specifically bind an analyte. The immunoassay is characterized by the use of specific binding properties of at least one particular antibody to isolate, target or quantify the analyte.

In particular embodiments, the immunoassay comprises the steps of: (a) depositing a biological sample on a support having immobilized bound anti-PCSK9 antibody AX213 bound thereto; (b) contacting the support having the biological sample deposited thereon with anti-PCSK9 antibody AX1 bearing a detectable label; and (c) detecting the label.

PCSK9 refers to proprotein convertase subtilisin-kexin type 9 (PCSK9), also known as neural apoptosis-regulated convertase 1 (NARC-1), a proteinase K-like subtilase identified as the 9^{th} member of the secretory subtilase family (Seidah, N. G., et al., 2003 PROC NATL ACAD SCI USA 100:928-933), as defined in the literature and, unless otherwise stated, includes both the soluble and insoluble forms. The term may in appropriate context refer to either an antigenic component thereof or the genetic locus.

AX213 is an antibody molecule comprising a variable light (“VL”) sequence comprising SEQ ID NO: 3 and a variable heavy (“VH”) sequence comprising SEQ ID NO: 7. In particular embodiments, AX213 is a full length antibody molecule. In specific embodiments, AX213 is an IgG antibody molecule, and in particular embodiments, an IgG2. In specific embodiments, AX213 comprises (a) light chain comprising SEQ ID NO: 1 or SEQ ID NO: 11 and (b) a heavy chain comprising SEQ ID NO: 9.

AX1 is an antibody molecule comprising a variable light (“VL”) sequence comprising SEQ ID NO: 15 and a variable heavy (“VH”) sequence comprising SEQ ID NO: 19. In particular embodiments, AX1 is a full length antibody molecule. In specific embodiments, AX213 is an IgG antibody molecule, and in particular embodiments, an IgG2. In specific embodiments, AX213 comprises (a) light chain comprising SEQ ID NO: 13 or SEQ ID NO: 23 and (b) a heavy chain comprising SEQ ID NO: 21.

Antibody molecules can exist, for example, as intact immunoglobulins or as a number of well characterized fragments produced by, for example, digestion with various peptidases. The recognized immunoglobulin genes include the kappa, lambda, alpha, gamma, delta, epsilon and mu constant region genes, as well as a myriad of immunoglobulin variable region genes. Light chains are classified as gamma, mu, alpha, delta, or epsilon, which in turn define the immunoglobulin classes, IgG, IgM, IgA, IgD and IgE, respectively. “Whole” antibodies or “full length” antibodies often refers to proteins that comprise two heavy (H) and two light (L) chains inter-connected by disulfide bonds which comprise: (1) in terms of the heavy chains, a variable region (abbreviated herein as “V_{H}”) and a heavy chain constant region which comprises three domains, C_{H1}, C_{H2}, and C_{H3}; and (2) in terms of the light chains, a light chain variable region (abbreviated herein as “V_{L}”) and a light chain constant region which comprises one domain, C_{L}. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially a Fab with part of the hinge region broken. While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

In specific embodiments, the AX213 and AX1 antibody molecules are, independently, isolated prior to use. “Isolated”, as used herein, refers to a property that makes them different from that found in nature. The difference can be, for example, that they are of a different purity than that found in nature, or that they are of a different structure or form part of a different structure than that found in nature. A structure not found in nature, for example, includes recombinant human immunoglobulin structures. Other examples of structures not found in nature are antibody molecules substantially free of other cellular material.

A detectable label, as used herein, refers to another molecule or agent incorporated into or affixed to the antibody molecule. In one embodiment, the label is a detectable marker, e.g., a radiolabeled amino acid or attachment to a polypeptide of biotinyl moieties that can be detected by marked avidin (e.g., streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, and analogs or homologs thereof. In some embodiments, labels are attached by spacer arms of various lengths to reduce potential steric hindrance.

In particular embodiments of the present invention, the immunoassay is a solid phase immunoassay. In specific embodiments, the solid phase immunoassay is a dissociation-enhanced lanthanide fluorescence immunoassay (DELFIA). However, it is within the scope of the current invention to use any solution-based or solid phase immunoassay as will be well familiar to those of skill in the art. Such assays include, without limitation, assays using magnetic beads as labels in lieu of enzymes, ELISAs, radioisotopes, or fluorescent moieties (fluorescent immunoassays).

The biological sample is selected from the group consisting of blood, plasma and serum. In particular embodiments, the blood, plasma and serum are derived from a mammalian subject including but not limited to humans.

The present invention further relates to a method for measuring PCSK9 in the presence of a putative PCSK9 antagonist. Said method comprises the steps of performing an immunoassay on a biological sample which has been contacted with a putative PCSK9 antagonist and comparing the level of PCSK9 in said sample against a standard having a known concentration of PCSK9. In particular embodiments, the method comprises (a) depositing the biological sample on a support having immobilized anti-PCSK9 antibody AX213; (b) contacting the support having the biological sample deposited thereon with anti-PCSK9 antibody AX1 bearing a detectable label; (c) detecting the label; and (d) comparing the level of PCSK9 in said sample against a standard having a known concentration of PCSK9. In a preferred embodiment, the immunoassay is a solid phase immunoassay. In a more preferred embodiment, the solid phase immunoassay is a dissociation-enhanced lanthanide fluorescence immunoassay (DELFIA).

The anti-PCSK9 immobilized antibody AX213, in specific embodiments, is coated on plates (in particular embodiments, black high binding assay plates) overnight. In particular embodiments, black high binding assay plates are coated overnight at 4° C. with 100-500 ng/well of AX213 antibody.

The biological sample is selected from the group consisting of blood, plasma and serum. In particular embodiments, the blood, plasma and serum are derived from a mammalian subject including but not limited to humans.

In particular embodiments, 10-50 ng/well of biotinylated AX1IgG is used for antigen detection.

Use of the term “antagonist” or derivatives thereof (e.g., “antagonizing”) refers to the fact that the subject molecule or agent can antagonize, oppose, counteract, inhibit, neutralize, or curtail the functioning of PCSK9. In specific embodiments, the antagonist reduces the functioning or activity or PCSK9 by at least 10%, or at least 20%, 30%, 40%, 50%, 60%, 70%, 80%, 90%, or 95%. Reference herein to PCSK9 function or PCSK9 activity refers to any function or activity that is driven by, requires, or is exacerbated or enhanced by PCSK9.

The present invention additionally relates to a kit for measuring circulating PCSK9 levels in a biological sample, comprising:

a). a biological sample collection device;

b). a composition comprising an immunoassay which comprises a coating or capture antibody and a detection antibody;

and c). a means for detecting a reaction between PCSK9 antigen in the sample and antibodies in the immunoassay; wherein the coating or capture antibody is AX213 and the detecting antibody is AX1.

In particular embodiments, the kit comprises the AX213 antibody immobilized on a support.

Kits typically but need not include a label indicating the intended use of the contents of the kit. The term label in the context of the kit includes any writing, or recorded material supplied on or with the kit, or which otherwise accompanies the kit.

The examples below are provided to illustrate the present invention without limiting the same hereto. The following list of acronyms are employed therein:

BSA: bovine serum albumin
ddH20 double distilled water

### EDTA: Ethylenediaminetetraacetic Acid

### IPTG: Isopropyl-Beta-d-Thiogalactopyranoside

PBS: Phosphate-buffered saline
PBST or PBS-T: Phosphate-buffered saline containing Tween
TBS-T: Tris-buffered saline containing Tween

### Example 1

### PCSK9 Antagonists AX213 & AX1

The PCSK9 antagonists used in this assay are antibodies AX213 and AX1. AX213 and AX1 are disclosed in copending applications Ser. Nos. 61/256,732 and 61/256,720 filed Oct. 30, 2009, which are incorporated in their entirety herein.

PDL1 Phage Library Panning Against PCSK9 Protein: AX1 and AX213 were identified by panning the VH3/Vκ3 and VH3/Vκ1 PDL1 Abmaxis synthetic human Fab libraries against human PCSK9. Antigen protein PCSK9 was coated on Maxisorp well stripe (Nunc-Immuno Modules) at a concentration of 1-10 μg/ml for overnight at 4° C. Multiple wells of antigen were prepared for each library. 5% milk in PBS was used to block the coated wells at room temperature for 1-2 hours. After a wash with PBS, 100 μl of phage library solution/well (usually 1−5×10¹² in 2% milk-PBS) was added into 4 parallel wells, and incubated for designed length of time (usually 1-2 hours). After several washings with PBST and PBS, the bound phages were eluted from the wells with fresh-prepared 1.4% triethylamine in ddH20 (10 minutes incubation at room temperature), followed immediately with neutralization by adding 50 μl of 1M Tris-HCl (pH 6.8).

The eluted, enriched phage pool was further amplified through the following steps: First, TG1 cells were infected with eluted phages at 37° C. for 1 hour, then plated out on 2YT agar plates with 2% glucose and 100 μg/ml carbenicillin for overnight culture. Thus TG1 cells harboring enriched phagemid library were harvested from the plates, and infected with helper phage GMCT for 1 hour. The Fab-display phages were then generated from those TG1 cells harboring both library phagemids and GMCT helper phage genome by overnight growth in 2xYT/carbenicillin/Kanamycin at 22° C. The phagemid particles were purified from overnight culture supernatants by precipitation with PEG/NaCl, and re-suspended in PBS. The PEG-precipitation was repeated once. The phage concentration was determined by OD₂₆₈ measurement.

With amplified first round phages, the panning process as described above was repeated twice for further enrichment of PCSK9-binding phages. The eluted phages from the third round panning were used to infect TG1 cells. The TG1 cells harboring phagemids from third round panning were picked from 2YT agar plates for Fab ELISA screening assay.

Fab ELISA Screening For PCSK9 Binders: Over 10,000 clones from third round panning were picked by MegaPix Picking Robot (Genetix), and inoculated into 384-well plates with 60 μl of 2YT/2% Glucose/carbenicillin for overnight culture at 30° C. with 450 rpm shaking. The duplicated plates were made by transferring ˜1-3 μl overnight culture from each well into new plates with 50 μl/well of 2YT/0.1% Glucose/carbenicillin. The duplicated plates were incubated in a shaker at 30° C. for 6 hours, then 10 μl/well of IPTG was added for a final concentration of 1 mM. After overnight culture at 22° C., the soluble Fab in IPTG-induction plates were released by adding lysozyme into each well.

To detect the antigen binding activity of soluble Fabs generated from the above experiment, the antigen plates were generated by overnight coating of 5 μg/ml antigen. After blocking with milk-PBS and a wash with PBST, 15-20 μl of Fab samples from IPTG-induction plates was transferred into antigen plates for 1-2 hours incubation at room temperature. The plates were washed 5 times with PBS-T, and added with 1:2000 diluted goat anti-human Kappa-HRP (SouthernBiotech Cat. No. 2060-05) or 1:10,000 diluted goat anti-human Fab-HRP in 5% MPBS for 1 hour incubation. After washing away unbound HRP-conjugates with PBST, the substrate solution QuantaBlu WS (Pierce 15169) was then added to each well and incubated for 5-15 minutes. The relative fluorescence units (RFU) of each well was measured to determine the Fab binding activity by using excitation wavelength 330 nm and emission detection wavelength 410 nm.

The ELISA results showed 30 to 80% clones from third round panning of individual PDL1 sun-libraries bound to antigen PCSK9. The positive clones were then sent out for DNA sequencing.

The sequences are set forth as follows:

AX213
AX213 FULL LIGHT CHAIN PROTEIN
[SEQ ID NO: 1]
EIVLTQSPATLSLSPGERATITCRASQYVGSYLNWYQQKPGQAPRLLIYDASNRATGIPAR
FSGSGSGTDFTLTISSLEPEDFAVYYCQVWDSSPPVVFGGGTKVETKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
AX213 FULL LIGHT CHAIN NUCLEIC ACID
[SEQ ID NO: 2]
GAAATCGTGCTGACCCAGTCTCCAGCCACCCTGTCTCTGTCTCCCGGGGAACGTGCC
ACCATCACCTGCCGTGCCTCTCAGTATGTCGGCAGCTACCTGAACTGGTATCAGCAG
AAGCCAGGTCAGGCGCCACGTCTGCTGATCTACGACGCCTCTAACCGTGCCACCGGT
ATCCCAGCCCGTTTCTCTGGTTCTGGTTCTGGCACCGACTTCACCCTGACCATCTCTT
CTCTGGAACCAGAAGACTTCGCCGTGTACTACTGCCAGGTATGGGACAGCTCTCCTC
CTGTGGTGTTCGGTGGTGGTACCAAAGTGGAAATCAAGCGTACGGTGGCTGCACCAT
CTGTATTCATCTTCCCGCCATCTGATGAGCAGTTGAAATCTGGAACTGCCTCTGTTGT
GTGCCTGCTGAATAACTTCTATCCCAGAGAGGCCAAAGTACAGTGGAAGGTGGATA
ACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACA
CAAAGTCTACGCCTGCGAAGTCACCCATCAGGGCCTGAGCTCGCCCGTCACAAAGA
GCTTCAACAGGGGAGAGTGT
AX213-VL,
[SEQ ID NO: 3]
CDRs underlined
EIVLTQSPATLSLSPGERATITCRASQYVGSYLNWYQQKPGQAPRLLIYDASNRATGIPAR
FSGSGSGTDFTLTISSLEPEDFAVYYCQVWDSSPPVVFGGGTKVEIK
AX213-VL
[SEQ ID NO: 4]
GAAATCGTGCTGACCCAGTCTCCAGCCACCCTGTCTCTGTCTCCCGGGGAACGTGCC
ACCATCACCTGCCGTGCCTCTCAGTATGTCGGCAGCTACCTGAACTGGTATCAGCAG
AAGCCAGGTCAGGCGCCACGTCTGCTGATCTACGACGCCTCTAACCGTGCCACCGGT
ATCCCAGCCCGTTTCTCTGGTTCTGGTTCTGGCACCGACTTCACCCTGACCATCTCTT
CTCTGGAACCAGAAGACTTCGCCGTGTACTACTGCCAGGTATGGGACAGCTCTCCTC
CTGTGGTGTTCGGTGGTGGTACCAAAGTGGAGATCAAA
AX213 FD CHAIN (FOR FABS) PROTEIN
[SEQ ID NO: 5]
QVQLLESGGGLVQPGGSLRLSCKASGYTFSRYGINWVRQAPGKGLEWIGRIDPGNGGTR
YNEKFKGKATISRDNSKNTLYLQMNSLRAEDTAVYYCARANDGYSFDYWGQGTLVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHT
AX213 FD CHAIN (FOR FABS) NUCLEIC ACID
[SEQ ID NO: 6]
caggtgcaattgctggaatctggtggtggtctggtgcagccaggtggttctctgcgtctgtcttgcaaggctagcggttacaccttctctcgcta
cggtatcaactgggtgcgtcaggcaccaggtaagggtctggaatggatcggtcggatcgacccaggtaacggtggtactaggtacaacgaa
aagttcaagggtaaggccaccatctctagagacaactctaagaacaccctgtacttgcagatgaactctctgcgtgccgaggacactgcagtg
tactactgcgcccgtgcaaatgacggttactccttcgactactggggtcagggtacgctggtgactgtctcgagcgcaagcaccaaaggccc
atcggtattccccctggcaccctcctccaagagcacctctgggggcacagcggccctgggctgcctggtcaaggactacttccccgagccg
gtgacggtgtcgtggaactcaggcgctctgaccagcggcgtgcacaccttcccggctgtcctacagtcctcaggactctactccctcagcag
cgtggtgactgtgccctccagcagcttgggcacccagacctacatctgcaacgtgaatcacaagcccagcaacactaaggtggacaagaaa
gttgagcccaaatcttgtgacaaaactcacaca
AX213-VH,
[SEQ ID NO: 7]
CDRS underlined
EVQLLESGGGLVQPGGSLRLSCKASGYTFSRYGINWVRQAPGKGLEWIGRIDPGNGGTR
YNEKFKGKATISRDNSKNTLYLQMNSLRAEDTAVYYCARANDGYSFDYWGQGTLVTV
SS
AX213-VH
[SEQ ID NO: 8]
CAGGTGCAATTGCTGGAATCTGGTGGTGGTCTGGTGCAGCCAGGTGGTTCTCTGCGT
CTGTCTTGCAAGGCTAGCGGTTACACCTTCTCTCGCTACGGTATCAACTGGGTGCGT
CAGGCACCAGGTAAGGGTCTGGAATGGATCGGTCGGATCGACCCAGGTAACGGTGG
TACTAGGTACAACGAAAAGTTCAAGGGTAAGGCCACCATCTCTAGAGACAACTCTA
AGAACACCCTGTACTTGCAGATGAACTCTCTGCGTGCCGAGGACACTGCAGTGTACT
ACTGCGCCCGTGCAAATGACGGTTACTCCTTCGACTACTGGGGTCAGGGTACGCTGG
TGACTGTCTCGAGC
AX213 IGG2 HEAVY CHAIN PROTEIN
[SEQ ID NO: 9]
EVQLLESGGGLVQPGGSLRLSCKASGYTFSRYGINWVRQAPGKGLEWIGRIDPGNGGTR
YNEKFKGKATISRDNSKNTLYLQMNSLRAEDTAVYYCARANDGYSFDYWGQGTLVTV
SSASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVAGPS
VFLFPPKPKDILMISRTPEVTCVVVDVSHEDPEVQFNWYVDGVEVHNAKTKPREEQFNS
TFRVVSVLTVVHQDWLNGKEYKCKVSNKGLPAPIEKTISKTKGQPREPQVYTLPPSREE
MTKNQVSLTCLVKGFYPSDIAVEWESNGQPENNYKTTPPMLDSDGSFFLYSKLTVDKSR
WQQGNVFSCSVMHEALHNHYTQKSLSLSPGK
AX213 IGG2 HEAVY CHAIN NUCLEIC ACID
[SEQ ID NO: 10]
GAGGTCCAACTTTTGGAGTCTGGAGGAGGACTGGTCCAACCTGGAGGCTCCCTGAG
ACTGTCCTGTAAGGCATCTGGCTACACCTTCAGCAGATATGGCATCAACTGGGTGAG
ACAGGCTCCTGGCAAGGGATTGGAGTGGATTGGCAGGATTGACCCTGGCAATGGAG
GCACCAGATACAATGAGAAGTTCAAGGGCAAGGCTACCATCAGCAGGGACAACAGC
AAGAACACCCTCTACCTCCAAATGAACTCCCTGAGGGCTGAGGACACAGCAGTCTA
CTACTGTGCCAGGGCTAATGATGGCTACTCCTTTGACTACTGGGGACAAGGCACCCT
GGTGACAGTGTCCTCTGCTAGCACCAAGGGCCCATCGGTCTTCCCCCTGGCGCCCTG
CTCCAGGAGCACCTCCGAGAGCACAGCGGCCCTGGGCTGCCTGGTCAAGGACTACT
TCCCCGAACCGGTGACGGTGTCGTGGAACTCAGGCGCTCTGACCAGCGGCGTGCAC
ACCTTCCCGGCTGTCCTACAGTCCTCAGGACTCTACTCCCTCAGCAGCGTGGTGACC
GTGCCCTCCAGCAACTTCGGCACCCAGACCTACACCTGCAACGTAGATCACAAGCCC
AGCAACACCAAGGTGGACAAGACAGTTGAGCGCAAATGTTGTGTCGAGTGCCCACC
GTGCCCAGCACCACCTGTGGCAGGACCGTCAGTCTTCCTCTTCCCCCCAAAACCCAA
GGACACCCTCATGATCTCCCGGACCCCTGAGGTCACGTGCGTGGTGGTGGACGTGAG
CCACGAAGACCCCGAGGTCCAGTTCAACTGGTACGTGGACGGCGTGGAGGTGCATA
ATGCCAAGACAAAGCCACGGGAGGAGCAGTTCAACAGCACGTTCCGTGTGGTCAGC
GTCCTCACCGTCGTGCACCAGGACTGGCTGAACGGCAAGGAGTACAAGTGCAAGGT
CTCCAACAAAGGCCTCCCAGCCCCCATCGAGAAAACCATCTCCAAAACCAAAGGGC
AGCCCCGAGAACCACAGGTGTACACCCTGCCCCCATCCCGGGAGGAGATGACCAAG
AACCAGGTCAGCCTGACCTGCCTGGTCAAAGGCTTCTACCCCAGCGACATCGCCGTG
GAGTGGGAGAGCAATGGGCAGCCGGAGAACAACTACAAGACCACACCTCCCATGCT
GGACTCCGACGGCTCCTTCTTCCTCTACAGCAAGCTCACCGTGGACAAGAGCAGGTG
GCAGCAGGGGAACGTCTTCTCATGCTCCGTGATGCATGAGGCTCTGCACAACCACTA
CACACAGAAGAGCCTCTCCCTGTCTCCGGGTAAA
AX213 FULL LIGHT CHAIN PROTEIN
[SEQ ID NO: 11]
EIVLTQSPATLSLSPGERATITCRASQYVGSYLNWYQQKPGQAPRLLIYDASNRATGIPAR
FSGSGSGTDFTLTISSLEPEDFAVYYCQVWDSSPPVVFGGGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
AX213 IGG LIGHT CHAIN PAIRED WITH IGG2 NUCLEIC ACID
[SEQ ID NO: 12]
GAGATTGTGCTGACCCAGAGCCCTGCCACCCTGTCCCTGAGCCCTGGAGAGAGGGC
TACCATCACTTGTAGGGCAAGCCAATATGTGGGCTCCTACCTGAACTGGTATCAACA
GAAGCCTGGACAAGCCCCAAGACTGCTGATTTATGATGCCAGCAACAGGGCTACAG
GCATCCCTGCCAGGTTCTCTGGCTCTGGCTCTGGCACAGACTTCACCCTGACCATCTC
CTCCTTGGAACCTGAGGACTTTGCTGTCTACTACTGTCAGGTGTGGGACTCCAGCCC
TCCTGTGGTGTTTGGAGGAGGCACCAAGGTGGAGATTAAGCGTACGGTGGCTGCAC
CATCTGTCTTCATCTTCCCGCCATCTGATGAGCAGTTGAAATCTGGAACTGCCTCTGT
TGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTCACAA
AGAGCTTCAACAGGGGAGAGTGT
AX1
AX1 FULL LIGHT CHAIN PROTEIN
[SEQ ID NO: 13]
DIQMTQSPSSLSASVGDRVTITCRASQDISRYLAWYQQKPGKAPKLLIYAASSLQSGVPS
RFSGSGSGTDFTLTISSLQPEDFATYYCAAYDYSLGGYVFGDGTKVEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
AX1 FULL LIGHT CHAIN NUCLEIC ACID
[SEQ ID NO: 14]
GACATCCAGATGACCCAGTCTCCATCTTCTCTGTCTGCCTCTGTGGGCGACCGGGTG
ACCATCACCTGCCGTGCCTCTCAGGATATCTCTAGGTATCTGGCCTGGTATCAGCAG
AAGCCAGGTAAGGCGCCAAAGCTGCTGATCTACGCCGCCTCTTCTTTGCAGTCTGGT
GTGCCATCTCGTTTCTCTGGTTCTGGTTCTGGCACCGACTTCACCCTGACCATCTCTT
CTTTGCAGCCAGAAGACTTCGCCACCTACTACTGCGCGGCTTACGACTATTCTTTGG
GCGGTTACGTGTTCGGTGATGGTACCAAAGTGGAGATCAAACGTACGGTGGCTGCA
CCATCTGTCTTCATCTTCCCGCCATCTGATGAGCAGTTGAAATCTGGAACTGCCTCTG
TTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTCACAA
AGAGCTTCAACAGGGGAGAGTGT
AX1-VL,
[SEQ ID NO: 15]
CDRs underlined
DIQMTQSPSSLSASVGDRVTITCRASQDISRYLAWYQQKPGKAPKLLIYAASSLQSGVPS
RFSGSGSGTDFTLTISSLQPEDFATYYCAAYDYSLGGYVFGDGTKVEIK
AX1-VL
[SEQ ID NO: 16]
GACATCCAGATGACCCAGTCTCCATCTTCTCTGTCTGCCTCTGTGGGCGACCGGGTG
ACCATCACCTGCCGTGCCTCTCAGGATATCTCTAGGTATCTGGCCTGGTATCAGCAG
AAGCCAGGTAAGGCGCCAAAGCTGCTGATCTACGCCGCCTCTTCTTTGCAGTCTGGT
GTGCCATCTCGTTTCTCTGGTTCTGGTTCTGGCACCGACTTCACCCTGACCATCTCTT
CTTTGCAGCCAGAAGACTTCGCCACCTACTACTGCGCGGCTTACGACTATTCTTTGG
GCGGTTACGTGTTCGGTGATGGTACCAAAGTGGAGATCAAA
AX1 FD CHAIN (FOR FABS) PROTEIN
[SEQ ID NO: 17]
EVQLLESGGGLVQPGGSLRLSCKASGFTFTSYYMHWVRQAPGKGLEWIGRINPDSGSTK
YNEKFKGRATISRDNSKNTLYLQMNSLRAEDTAVYYCARGGRLSWDFDVWGQGTLVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHT
AX1 FD CHAIN (FOR FABS) NUCLEIC ACID
[SEQ ID NO: 18]
gaagtgcagctgctggaatctggtggtggtctggtgcagccaggtggttctctgcgtctgtcttgcaaggcctctggtttcaccttcacttcttac
tacatgcactgggtgcgtcaggcaccaggtaagggtctggaaiggatcggtcggatcaacccagattctggtagtactaagtacaacgagaa
gttcaagggtcgtgccaccatctctagagacaactctaagaacaccctgtacttgcagatga.actctctgcgtgccgaggacactgcaggta
ctactgcgcccgtggtggtcgtttatcctgggacttcgacgtctggggtcagggtacgaggtgactgtctcgagcgcaagcaccaaaggcc
catcggtattccccctggcaccctcctccaagagcacctctgggggcacagcggccctgggctgcctggtcaaggactacttccccgagcc
ggtgacggtgtcgtggaactcaggcgctctgaccagcggcgtgcacaccttcccggctgtcctacagtcctcaggactctactccctcagca
gcgtggtgactgtgccctccagcagcttgggcacccagacctacatctgcaacgtgaatcacaagcccagcaacactaaggtggacaagaa
agttgagcccaaatcttgtgacaaaactcacaca
AX1-VH,
[SEQ ID NO: 19]
CDRs underlined
EVQLLESGGGLVQPGGSLRLSCKASGFTFTSYYMHWVRQAPGKGLEWIGRINPDSGSTK
YNEKFKGRATISRDNSKNTLYLQMNSLRAEDTAVYYCARGGRLSWDFDVWGQGTLVT
VSS
AX1-VH
[SEQ ID NO: 20]
GAAGTGCAGCTGCTGGAATCTGGTGGTGGTCTGGTGCAGCCAGGTGGTTCTCTGCGT
CTGTCTTGCAAGGCCTCTGGTTTCACCTTCACTTCTTACTACATGCACTGGGTGCGTC
AGGCACCAGGTAAGGGTCTGGAATGGATCGGTCGGATCAACCCAGATTCTGGTAGT
ACTAAGTACAACGAGAAGTTCAAGGGTCGTGCCACCATCTCTAGAGACAACTCTAA
GAACACCCTGTACTTGCAGATGAACTCTCTGCGTGCCGAGGACACTGCAGTGTACTA
CTGCGCCCGTGGTGGTCGTTTATCCTGGGACTTCGACGTCTGGGGTCAGGGTACGCT
GGTGACTGTCTCGAGC
AX1 IGG2 HEAVY CHAIN PROTEIN
[SEQ ID NO: 21]
EVQLLESGGGLVQPGGSLRLSCKASGFTFTSYYMHWVRQAPGKGLEWIGRINPDSGSTK
YNEKFKGRATISRDNSKNTLYLQMNSLRAEDTAVYYCARGGRLSWDFDVWGQGTLVT
VSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCCVECPPCPAPPVAG
PSVFLFPPKPKDTLMISRTPEVTCVVVDVSHEDPEVQFNWYVDGVEVIINAKTKPREEQF
NSTFRVVSVLTVVHQDWLNGKEYKCKVSNKGLPAPIEKTISKTKGQPREPQVYTLPPSRE
EMTKNQVSLTCLVKGFYPSDIAVEWESNGQPENNYKTTPPMLDSDGSFFLYSKLTVDKS
RWQQGNVFSCSVMHEALHNHYTQKSLSLSPGK
AX1 IGG2 HEAVY CHAIN NUCLEIC ACID
[SEQ ID NO: 22]
GAGGTCCAACTTTTGGAGTCTGGAGGAGGACTGGTCCAACCTGGAGGCTCCCTGAG
ACTGTCCTGTAAGGCATCTGGCTTCACCTTCACCTCCTACTATATGCACTGGGTGAG
ACAGGCTCCTGGCAAGGGATTGGAGTGGATTGGCAGGATAAACCCTGACTCTGGCA
GCACCAAATACAATGAGAAGTTCAAGGGCAGGGCTACCATCAGCAGGGACAACAGC
AAGAACACCCTCTACCTCCAAATGAACTCCCTGAGGGCTGAGGACACAGCAGTCTA
CTACTGTGCCAGGGGAGGCAGACTGTCCTGGGACTTTGATGTGTGGGGACAAGGCA
CCCTGGTGACAGTGTCCTCTGCTAGCACCAAGGGCCCATCGGTCTTCCCCCTGGCGC
CCTGCTCCAGGAGCACCTCCGAGAGCACAGCGGCCCTGGGCTGCCTGGTCAAGGAC
TACTTCCCCGAACCGGTGACGGTGTCGTGGAACTCAGGCGCTCTGACCAGCGGCGTG
CACACCTTCCCGGCTGTCCTACAGTCCTCAGGACTCTACTCCCTCAGCAGCGTGGTG
ACCGTGCCCTCCAGCAACTTCGGCACCCAGACCTACACCTGCAACGTAGATCACAA
GCCCAGCAACACCAAGGTGGACAAGACAGTTGAGCGCAAATGTTGTGTCGAGTGCC
CACCGTGCCCAGCACCACCTGTGGCAGGACCGTCAGTCTTCCTCTTCCCCCCAAAAC
CCAAGGACACCCTCATGATCTCCCGGACCCCTGAGGTCACGTGCGTGGTGGTGGAC
GTGAGCCACGAAGACCCCGAGGTCCAGTTCAACTGGTACGTGGACGGCGTGGAGGT
GCATAATGCCAAGACAAAGCCACGGGAGGAGCAGTTCAACAGCACGTTCCGTGTGG
TCAGCGTCCTCACCGTCGTGCACCAGGACTGGCTGAACGGCAAGGAGTACAAGTGC
AAGGTCTCCAACAAAGGCCTCCCAGCCCCCATCGAGAAAACCATCTCCAAAACCAA
AGGGCAGCCCCGAGAACCACAGGTGTACACCCTGCCCCCATCCCGGGAGGAGATGA
CCAAGAACCAGGTCAGCCTGACCTGCCTGGTCAAAGGCTTCTACCCCAGCGACATC
GCCGTGGAGTGGGAGAGCAATGGGCAGCCGGAGAACAACTACAAGACCACACCTCC
CATGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAA
CCACTACACACAGAAGAGCCTCTCCCTGTCTCCGGGTAAA
AX1 FULL LIGHT CHAIN PROTEIN
[SEQ ID NO: 23]
DIQMTQSPSSLSASVGDRVTITCRASQDISRYLAWYQQKPGKAPKLLIYAASSLQSGVPS
RFSGSGSGTDFTLTISSLQPEDFATYYCAAYDYSLGGYVFGDGTKVEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
AX1 IGG LIGHT CHAIN PAIRED WITH IGG2 NUCLEIC ACID
[SEQ ID NO: 24]
GACATCCAGATGACCCAGAGCCCATCCTCCCTGTCTGCCTCTGTGGGAGACAGGGTG
ACCATCACTTGTAGGGCAAGCCAGGACATCAGCAGATACCTGGCTTGGTATCAACA
GAAGCCTGGCAAGGCTCCAAAACTGCTGATTTATGCTGCCTCCTCCCTCCAATCTGG
AGTGCCAAGCAGGTTCTCTGGCTCTGGCTCTGGCACAGACTTCACCCTGACCATCTC
CTCCCTCCAACCTGAGGACTTTGCCACCTACTACTGTGCTGCCTATGACTACTCCCTG
GGAGGCTATGTGTTTGGAGATGGCACCAAGGTGGAGATTAAGCGTACGGTGGCTGC
ACCATCTGTCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAA
GGACAGCACCTACAGCCTCAGCAGCACCCTGACGCTGAGCAAAGCAGACTACGAGA
AACACAAAGTCTACGCCTGCGAAGTCACCCATCAGGGCCTGAGCTCGCCCGTCACA
AAGAGCTTCAACAGGGGAGAGTGT

Fab Protein Expression And Purification From TG1 Cells: 50 ml of overnight cultures for individual clones in 2YT/2% glucose/Carbenicillin 100 μg/ml were grown in 37° C. shaker incubator. In the second day, 750 mL to 1 L of 2YT/0.1% glucose/100 μg/mL Carbenicillin was inoculated for each clone by transferring 5-10 ml of the overnight culture. The cultures were grown at 30° C. with shaking for approximately 3-4 hours until OD600 ˜1. IPTG was added to the culture to reach the final concentration of 0.1-0.5 mM. After overnight IPTG induction at 22° C., the cells pellets were collected by centrifugation at 10,000 rpm for 10-15 minutes, to proceed for periplasmic preparation.

Soluble Fabs were extracted from cell periplasm. The periplasmic preparation was performed as follows. The TG1 pellet was re-suspended in 20 mL pre-chilled PPB buffer (20% Sucrose+2 mM EDTA+30 mM Tris, pH=8), and incubated on ice for 1 hour. The supernatant with soluble Fab was collected by centrifugation. Subsequently, the cell pellet was further re-suspended in 20 mL pre-chilled 5 mM magnesium sulfate with 1 hour incubation on ice. Two supernatants were combined for further Fab purification.

The soluble Fab from the periplasmic extraction was purified using a HiTrap Protein G HP column (GE Healthcare). The column was initially equilibrated with equilibration buffer (PBS or Tris, pH 7.3). The supernatant from periplasmic preparation was loaded onto a 1-ml or 5-mL protein-G column (HiTrap, GE healthcare). After wash with 10 column volumes (CVs) of equilibration buffer, Fab protein was eluted with 8 CVs of elution buffer (0.3 M acetic acid, pH3). The eluted fractions were collected, and neutralized with 0.5 volume of 1M Tris, pH 9 buffer. The Fab samples were buffer-exchanged into PBS using Amicon centrifugal filters with 10 kD molecular weight cutoff. The quality of purified Fab was analyzed using size exclusion HPLC (SE-HPLC). Purified Fab was also used for ELISA assay and Biacore assay (below). Overall, the summary of Fab yields is ˜1-2 mg/L with high degree of variability, from less than 1 mg/L to well over 10 mg/L. All Fabs show single main peak by SE-HPLC. The ELISA assay results confirmed all Fab bound to human PCSK9 antigen.

Anti-PCSK9 Monoclonal Antibody Purification From Glycoengineered Pichia Pastoris: Anti-PCSK9 monoclonal antibody expressed in glyco-engineered Pichia pastoris GFI 5.0 host YGLY8316, which is capable of transferring terminal galactose at its complex N-linked glycan. Anti-PCSK9 heavy and light chains were codon optimized and expressed under methanol tightly inducible promoter AOX1 using Saccharomyces cerevisiae alpha mating factor presequence as secretion signal sequence. The glycoengineered Pichia strain producing this antibody was named as YGLY18513. Anti-PCSK9 antibody from YGLY18513 was captured from cell free supernatant media by affinity chromatography using MabSelect™ medium from GE Healthcare (Cat. #17-5199-01). The cell free supernatant was loaded on to Mabselect column (XK 16/20, 1.6 cm×10.0 cm) pre-equilibrated with three column volume of 20 mM Tris-HCl pH7.0 at a flow rate of 5.0 mL/min. The column was washed with three column volumes of the 20 mM Tris-HCl pH7.0 followed by a five column volume wash with 20 mM Tris-HCl pH7.0 containing 1M NaCl to remove the host cell proteins. The anti-PCSK9 antibody was eluted with five column volume of 100 mM Glycine, 100 mM Arginine pH 3.0 and immediately neutralized with 1M Tris-HCl pH8.0. Antibody was well expressed in Pichia.

Strong Cation Exchange Chromatography employing Source 30S resin from GE Healthcare (Cat #1117-1273-02) was used as the second step purification to remove the clipped species and aggregates. Mabselect pool of the anti-PCSK9 antibody was 5× diluted with 25 mM Sodium acetate pH5.0 and loaded on to the Source 30S column pre-equilibrated with three column volume of 25 mM Sodium acetate pH5.0. After loading, the column was washed with three column volume of the 25 mM Sodium acetate pH5.0 and elution was performed by developing a linear gradient over ten column volume ranging from 100 mM to 150 mM Sodium chloride in 25 mM Sodium acetate pH5.0. The fractions containing good assembled anti-PCSK9 antibody was pooled together. The Source30S pooled fractions that contained the anti-PCSK9 antibody was buffer exchanged into the formulation buffer containing 6% Sucrose, 100 mM Arginine, 100 mM Histidine pH6.0 (HyClone® Cat #RR10804.02) and sterile filtered using 0.2 μm PES (PolyEtherSulfone) membrane filter and stored @4° C. until release.

### Example 2

### Measurement of PCSK9 in Human EDTA Plasma

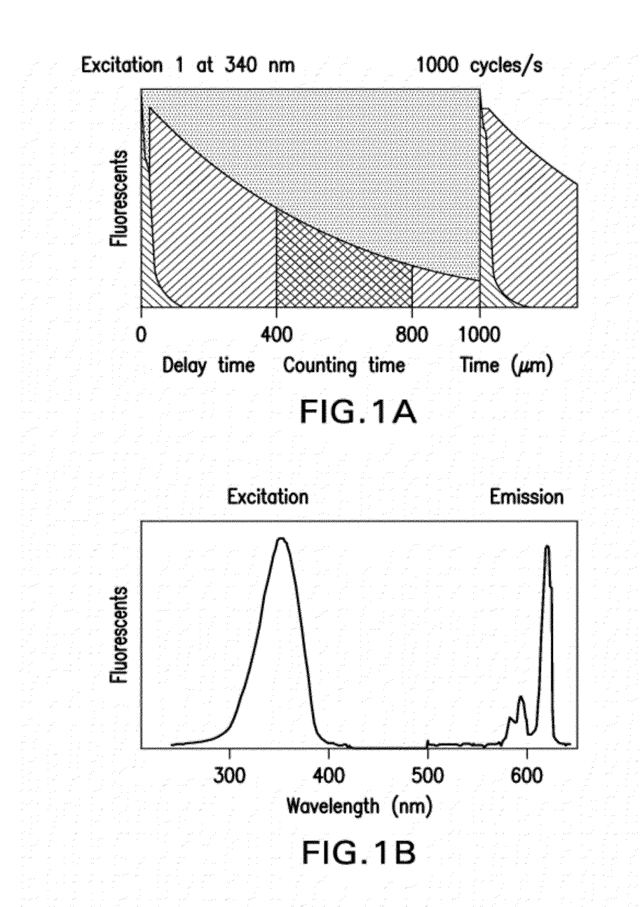

The assay employs a Dissociation-Enhanced Lanthanide Fluorescent Immunoassay (DELFIA) Time-Resolved Fluorometry (TRF) method. DELFIA TRF assays rely on the fluorescent properties of lanthanide chelate labels which allow for long fluorescence decay times and large Stokes' shifts; see FIGS. 1A-B. The long fluorescence decay times allow the user to measure fluorescence after background fluorescence has subsided, effectively reducing background emissions that normally accompany samples. In addition, the assay has a large Stokes shift (360 nM excitation/620 nM emission) which allow for clean peak fluorescence detection without interfering peaks and peak shoulders. These characteristics of DELFIA TRE effectively reduce background emission to a level that allows for increased measurement sensitivities.

The assay relies on the direct adsorption of a capture antibody onto the surface of a high binding Costar Plate. Samples, standards, and controls are added to the well followed by secondary antibody and after immunoreactions; the lanthanide label is dissociated from the complex in enhancement solution. The free lanthanide (Eu³⁺, Europium) rapidly forms a new highly fluorescent and stable chelate with the components of the enhancement solution. For analysis, plates are loaded into the Biotek Synergy 2 instrument and excited at a wavelength of 360 nm and the emission is read at 620 nm. The assay quantitatively measures the concentration of PCSK9 in human plasma.

Equipment: Biotek Synergy 2 Plate Reader (Excitation filter-360±40 nM, Emission filter-620±40 nM); Assorted pipettors; Vortex Mixer; Plate Shaker; Biohit Multichannel Pipettor (12004); Beckman Coulter Biomek FX; Boekel Jitterbug Model 130000

Supplies: Microplate Adhesive Film (USA Scientific cat #2920-0000); 1.5 mL microfuge tubes (Eppendorf, Cat #022363204); Black High Binding Assay Plate (Costar #3295); Pipet tips; EDTA Vacutainer Tubes for Plasma Collection (BD, cat #366643)

Reagents: (1) DELFIA Components (Perkin Elmer) [Streptavidin/Europium (100 μg/mL), stored at 4° C. (catalog #1244-360); DELFIA Assay Buffer, stored at 4° C. (catalog #1244-111); DELFIA Enhance, stored at 4° C. (catalog #1244-105)]; (2) Antibodies [AX213 (monoclonal Ab to human PCSK9) capture antibody, stored at 4° C. and AX1 (monoclonal Ab to human PCSK9) biotinylated secondary antibody, stored at 4° C.]; (3) Heterophilic Blocking Reagent 1 (HBR1, Purified), Scantibodies Laboratory, catalog #3KC533˜20 mg/mL; (4) 10% Tween-20 stored at room temperature (Bio-Rad, catalog #161-0781); (5) MSD Blocker A: stored at 4° C. (Meso Scale Discovery, catalog #R93AA-1); (6) TBS-T Wash Buffer (Sigma catalog #T-9039) [1 packet mixed into 1 liter Milli-Q grade water, Final concentration: 50 mM Tris-buffered saline, 0.05% Tween-20 in 1000 mL, stored at room temperature]; (7) 1× Phosphate Buffered Saline Solution (Fluka, catalog #79383) [5.0 mL 10×PBS was diluted into 45 mL Milli-Q grade water, Final concentration: 1×]; (8) Coating Solution [prepared immediately before use as follows: 5.00 μL of AX213 (stock=10.05 mg/mL) into 5995 μL of 1×PBS, Coat Solution is 8.375 μg/mL AX213]; (9) Blocking Solution [prepared day of experiment as follows: 900 mg of MSD Blocker A (BSA) into 30.0 mL of TBS-T Wash Buffer, Final Concentration: 3%]; (10) Assay Buffer (AB) [prepared day of experiment as follows in Table 1 below]:

TABLE 1

| Component | Volume | Final Concentration |
|---|---|---|
| Blocking Solution | 5000 μL | 1% BSA |
| TBS-T Wash Buffer | 9490 μL | — |
| 10% Tween-20 | 375 μL | 0.25% |
| HBR(18.44 mg/mL) | 138.8 μL | 30 μg HBR to 25 μL plasma |
| Total volume | 15000 μL | |

(11) 1% BSA [prepared day of experiment as follows: 4.0 mL Blocking Solution was pipetted into 8.0 mL TBS-T Wash Buffer]; (12) Biotinylated Secondary Antibody Solution [prepared immediately before use as follows: 5.5 μL AX1 Ab (stock=1.0 mg/mL) into 5494.5 μL 1% BSA, Final concentration: 1.0 μg/Ml]; and (12) Strep-Eu Solution [prepared immediately before use and protected from light, 8.0 μL Strep-Eu (stock=100 μg/mL) into 7992 μL DELFIA Assay Buffer, Final concentration: 0.100 μg/mL].

Preparation of Calibrator Curve: The master stock concentration of PCSK9 is 1.32 mg/mL. A 30 μg/mL stock was prepared using a 1:44 dilution with Assay Buffer from the master stock.

TABLE 2

| | | Volume | |
|---|---|---|---|
| Calibrator | | Assay | Dilution |
| (Nm) | Volume Calibrator/Stock | Buffer | Factor |
| 10.25 | 6.0 μL of 30 μg/mL Stock (384.4 nM) | 219.0 μL | 37.5 |
| 3.42 | 75 μL of 10.25 nM Calibrator | 150 μL | 3.0 |
| 1.14 | 75 μL of 3.42 nM Calibrator | 150 μL | 3.0 |
| 0.38 | 75 μL of 1.14 nM Calibrator | 150 μL | 3.0 |
| 0.13 | 75 μL of 0.38 nM Calibrator | 150 μL | 3.0 |
| 0.04 | 75 μL of 0.13 nM Calibrator | 150 μL | 3.0 |
| 0.014 | 75 μL of 0.04 nM Calibrator | 150 μL | 3.0 |
| 0.004 | 75 μL of 0.014 nM Calibrator | 150 μL | 3.0 |

Biomek FX Procedure: All calibrations of the Span-8 Head were specifically created for PCSK9. All robot pipetting functions were performed using the Span-8 Head. The program is divided into three sections: (1) Sample Dilution: 140 μl of assay buffer was added to each well in a polypropylene dilution plate; 20 μl, of each QC and clinical sample were added to the wells containing the assay buffer; (2) Sample Addition: Each QC and clinical sample in the dilution plate was mixed 3 times; 50 μL of each QC and clinical sample were added in duplicate to the Costar Assay Plate; and Standard Addition: 50 μL of each calibrator was added in duplicate to the Costar Assay Plate.

Biotek Synergy 2 Settings: Plate was shaken for 5 minutes on the lowest setting and then read. Excitation and Emission, 360 nm (40 nm range) and 620 nm (40 nm range), respectively. Delay Time is 250 μSec with a total count time of 1000 μSec.

Assay Procedure: (1) Plate Coating: 60 μL of Coating Solution was added per well, left at 4° C. overnight, and sealed with a plated sealer. (2) Blocking the Plate: Without washing the plate, 150 μL of Blocking Solution was added per well and incubated shaking for 1 hour at room temp. Jitterbug was turned on and temp. set to 37° C. (3) Recombinant PCSK9 Curve: 6.0 μL of the 30 μg/mL stock was added into 219.0 μL Assay Buffer, and 3-fold serial diluted using 75 μL calibrator into 150 μL Assay Buffer. (4) Sample and Calibrator Addition: After Blocking, plate was washed as described in step 1, and run on Biomek FX. The program diluted the samples and QCs 1:8 in Assay Buffer. Calibrators (standards) were not diluted. Final volume per well was 50 μL. Plate was then incubated in the Jitterbug for 1 hour shaking at 37° C. (5) Detection Antibody: 50 μL of the biotinylated secondary antibody solution was added to each well. Final Concentration of Antibody was 1.0 μg/mL. Plate was incubated 1 hour shaking at room temp. (6) Strep-Eu: 75 μL of the Strep-Eu solution was added to each well. Concentration of Strep-Ru was 0.10 μg/mL. Plate was incubated 20 min shaking at room temp. (7) Enhance Solution: 1004 of DELFIA Enhance solution was added to each well, and the plate covered with black lid and read on Biotek Synergy 2 Plate Reader. (8) Read plate: The DELFIA Program was run. Plate was shaked 5 minutes, then was read at an excitation of 360 nm and emission of 620 nm.

Calculations: All calculations were completed using the Gen5 Software. Concentrations of unknowns were derived from the calibrator curve in nM PCSK9.

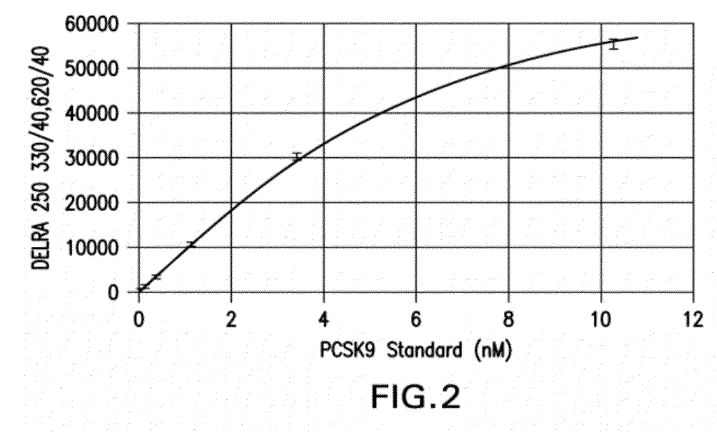

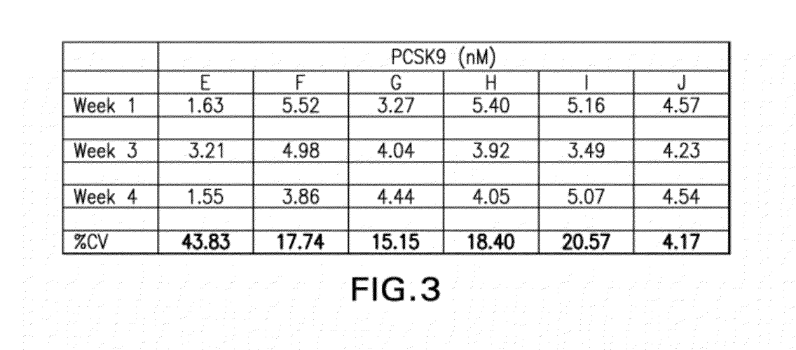

Results: FIG. 2 illustrates the recombinant human PCSK9 standard curve diluted in assay buffer. The range of the curve is 10.26 nM to 0.005 nM. FIG. 3 illustrates the biological variability of six normal healthy volunteers shown on three different days over three weeks. Concentration shown in nM.

What is claimed is:

1. A method of measuring circulating PCSK9 levels in a biological sample comprising the steps of performing an immunoassay on a biological sample obtained from a subject and comparing the level of PCSK9 in said sample against a standard having a known concentration of PCSK9, wherein a coating or capture antibody is AX213 and a detecting antibody is AX1.

2. The method of claim 1 wherein AX213 and AX1 are full length antibodies.

3. The method of claim 1 wherein AX213 comprises a variable light (“VL”) sequence comprising SEQ ID NO: 3 and a variable heavy (“VH”) sequence comprising SEQ ID NO: 7, and AX1 comprises a variable light (“VL”) sequence comprising SEQ ID NO: 15 and a variable heavy (“VH”) sequence comprising SEQ ID NO: 19.

4. The method of claim 1 wherein AX213 comprises a light chain comprising SEQ ID NO: 1 or SEQ ID NO: 11 and a heavy chain comprising SEQ ID NO: 9 and AX1 comprises (a) light chain comprising SEQ ID NO: 13 or SEQ ID NO :23 and (b) a heavy chain comprising SEQ ID NO: 21.

5. The method of claim 1 wherein performing an immunoassay comprises: (a) depositing a biological sample on a support having immobilized anti-PCSK9 antibody AX213; (b) contacting the support having the biological sample deposited thereon with anti-PCSK9 antibody AX1 bearing a detectable label; and (c) detecting the label.

6. The method of claim 1, wherein the immunoassay is a solid phase immunoassay.

7. The method of claim 6, wherein the solid phase immunoassay is a dissociation-enhanced lanthanide fluorescence immunoassay (DELFIA).

8. The method of claim 1, wherein said sample is selected from the group consisting of blood, plasma and serum.

9. The method of claim 8 wherein the blood, plasma or serum is from a human.

10. A method for performing an immunoassay on a biological sample which has been contacted with a putative PCSK9 antagonist which comprises (a) depositing the biological sample on a support having immobilized anti-PCSK9 antibody AX213; (b) contacting the support having the biological sample deposited thereon with anti-PCSK9 antibody AX1 bearing a detectable label; (c) detecting the label; and (d) comparing the level of PCSK9 in said sample against a standard having a known concentration of PCSK9.

11. The method of claim 10 wherein AX213 and AX1 are full length antibodies.

12. The method of claim 10 wherein AX213 comprises a variable light (“VL”) sequence comprising SEQ ID NO: 3 and a variable heavy (“VH”) sequence comprising SEQ ID NO: 7, and AX1 comprises a variable light (“VL”) sequence comprising SEQ ID NO: 15 and a variable heavy (“VH”) sequence comprising SEQ ID NO: 19.

13. The method of claim 10 wherein AX213 comprises a light chain comprising SEQ ID NO: 1 or SEQ ID NO: 11 and a heavy chain comprising SEQ ID NO: 9 and AX1 comprises (a) light chain comprising SEQ ID NO: 13 or SEQ ID NO: 23 and (b) a heavy chain comprising SEQ ID NO: 21.

14. The method of claim 10, wherein the immunoassay is a solid phase immunoassay.

15. The method of claim 14, wherein the solid phase immunoassay is a dissociation-enhanced lanthanide fluorescence immunoassay (DELFIA).

16. The method of claim 10, wherein said sample is selected from the group consisting of blood, plasma and serum.

17. The method of claim 16 wherein the blood, plasma or serum is from a human.

18. A kit for measuring circulating PCSK9 levels in a biological sample, comprising:
a). a biological sample collection device;
b). a composition comprising an immunoassay which comprises a coating or capture antibody and a detection antibody; and
c). a means for detecting a reaction between PCSK9 antigen in the sample and antibodies in the immunoassay;
wherein the coating or capture antibody is AX213 and the detecting antibody is AX1.

19. The kit of claim 18 wherein AX213 and AX1 are full length antibodies.

20. The method of claim 19 wherein AX213 comprises a variable light (“VL”) sequence comprising SEQ ID NO: 3 and a variable heavy (“VH”) sequence comprising SEQ ID NO: 7, and AX1 comprises a variable light (“VL”) sequence comprising SEQ ID NO: 15 and a variable heavy (“VH”) sequence comprising SEQ ID NO: 19.

21. The method of claim 20 wherein AX213 comprises a light chain comprising SEQ ID NO: 1 or SEQ ID NO: 11 and a heavy chain comprising SEQ ID NO: 9 and AX1 comprises (a) light chain comprising SEQ ID NO: 13 or SEQ ID NO: 23 and (b) a heavy chain comprising SEQ ID NO: 21.
